(19) United States
(12) Patent Application Publication
(10) Pub. No.: US 2021/0307409 A1
(43) Pub. Date: Oct. 7, 2021

(54) ELECTRONIC CIGARETTE AUTOMATIC CLEANING APPARATUS

(71) Applicants: Kwan Sung Lee, Seoul (KR); GENERAL NET CORP., Seoul (KR)

(72) Inventor: Kwan Sung Lee, Seoul (KR)

(21) Appl. No.: 17/267,135

(22) PCT Filed: May 8, 2019
(86) PCT No.: PCT/KR2019/005464

§ 371 Date: Feb. 9, 2021

(30) Foreign Application Priority Data
Aug. 10, 2018 (KR) 10-2018-0093695

(51) Int. Cl. A24F 40/85; B08B 3/12; B08B 9/023; B08B 9/032; B08B 13/00; B08B 3/08

(52) U.S. Cl. CPC A24F 40/85; B08B 3/12; B08B 9/023; B08B 2209/032; B08B 13/00; B08B 3/08; B08B 2209/005; B08B 9/032

(57) ABSTRACT

The present disclosure provides an electronic cigarette automatic cleaning apparatus includes a main body having an accommodating space, a cleaning tub provided in an accommodating space of the main body and storing a cleaning material for cleaning an electronic cigarette device, a cover coupled to the cleaning tub and having a through hole through which a cleaning portion of the electronic cigarette device is immersed in the cleaning material, and an ultrasound generation portion provided in the main body and configured to emit ultrasonic waves to the cleaning material to separate a foreign material from the electronic cigarette device.

### TECHNICAL FIELD

The present disclosure relates to electronic cigarette automatic cleaning apparatuses, and more particularly, to an electronic cigarette automatic cleaning apparatus capable of automatically, rapidly, and thoroughly cleaning the inside of an electronic cigarette device where foreign materials such as cigarette ash, cigarette powder, and dust are intensively accumulated, without using a special cleaning tool.

### BACKGROUND ART

In general, electronic cigarettes are products capable of replacing cigarettes by using nicotine concentrate.

Such electronic cigarettes are mainly used by smokers to quit smoking or to avoid the drawbacks of traditional cigarette smoking. Conventional electronic cigarettes work by evaporating the nicotine concentrate by using an atomizer, which the user then inhales. In the vaporization process of the nicotine concentrate, nicotine can be consumed without ingesting as many harmful products as are ingested when smoking cigarettes.

However, conventional electronic cigarettes using nicotine concentrate fail to satisfy many users because they lack the smell and taste of tobacco cigarettes.

In order to address such a problem, electronic cigarettes using tobacco cigarettes have been developed. One example is IQOS.

IQOS is used by inserting a heat stick such as a tobacco cigarette into an insertion tube of the IQOS device which heats the tobacco cigarette without burning it to release nicotine and other substances from the tobacco. The user inhales the released nicotine and other substances, which provides for a more satisfying experience due to the taste and aroma being more like a tobacco cigarette. It is also thought that by heating the tobacco instead of burning it, the harmful substances inhaled are reduced, because it operates in a steaming method rather than a method of burning the tobacco of the heat stick by using a heating unit protruding inside the insertion tube.

In electronic cigarettes using a heat stick as described above, various foreign substances such as cigarette ash and nicotine remain on the inner and outer surfaces of the insertion tube, the surface of the heating unit, and the bottom surface of the insertion tube after use, and thus regular cleaning is required.

Conventionally, these various foreign substances such as nicotine inside the insertion tube and the cover tube of IQOS were wiped and cleaned using a removal brush and a cotton swab provided when purchasing IQOS. This cleaning operation is very inconvenient and time-consuming, and also the insides of the insertion tube and the cover tube are not cleaned thoroughly because certain areas are difficult to reach with a cotton swab.

In addition, because the conventional removal brush can only coarsely remove deposits, undesirable or harmful materials remain on the insertion tube, the heating unit, and the bottom surface, and the remaining harmful materials are inhaled into the smoker's body and harm the smoker's health.

### PRIOR ART DOCUMENTS

### Patent Document

(Patent Document 1) Registered Utility Model No. 20-0469513 (Oct. 10, 2013)

### DISCLOSURE OF THE INVENTION

### Technical Problem

Provided is an electronic cigarette automatic cleaning apparatus capable of automatically and thoroughly cleaning the inside of an electronic cigarette device where foreign materials such as cigarette ash, cigarette powder, dust, and other undesirable materials are intensively accumulated, without using a special cleaning tool, and capable of completely removing harmful materials attached to the electronic cigarette device.

### Technical Solution

According to an aspect of the present disclosure, an electronic cigarette automatic cleaning apparatus includes a main body having an accommodating space, a cleaning tub provided in an accommodating space of the main body and storing a cleaning material for cleaning an electronic cigarette device, a cover coupled to the cleaning tub and having a through hole through which a cleanable portion of the electronic cigarette device is immersed in the cleaning material, and an ultrasound generation portion provided in the main body and configured to emit ultrasonic waves to the cleaning material to separate a foreign material from the electronic cigarette device.

A packing may be provided on an outer surface of the cleaning tub, and a fixing groove into which the packing is inserted and fixed may be formed in an inner surface of the cover.

A support step supporting the electronic cigarette device and having the through hole formed therein may be recessed in the cover, and a support groove for supporting a power button included in the electronic cigarette device is formed in an inner surface of the support step.

The electronic cigarette automatic cleaning apparatus may further include a charging portion provided in the main body and having a charging terminal to charge the ultrasound generation portion.

### Advantageous Effects

An electronic cigarette automatic cleaning apparatus according to the present disclosure may automatically, quickly, and thoroughly clean the inside and outside of an insertion tube, a heating portion, and a bottom surface of an electronic cigarette device where foreign materials such as cigarette powder, cigarette ash, and dust are intensively accumulated, thereby improving the convenience of a smoker who uses the electronic cigarette device. In addition, the electronic cigarette automatic cleaning apparatus according to the present disclosure may completely remove the foreign materials through a cleaning material instead of detaching foreign materials through a conventional foreign material removal brush, thereby enabling clean usage. Moreover, the electronic cigarette automatic cleaning apparatus according to the present disclosure may prevent a smoker from inhaling harmful ingredients generated from the cigarette powder, cigarette ash, or the like, thereby preventing an adverse influence upon the smoker's health.

### BRIEF DESCRIPTION OF THE DRAWINGS

FIG. 1 is a perspective view of an electronic cigarette automatic cleaning apparatus according to the present disclosure.

FIG. 2 is a view illustrating the inside of the electronic cigarette automatic cleaning apparatus according to the present disclosure.

FIGS. 3 and 4 are views illustrating states in which the electronic cigarette automatic cleaning apparatus according to the present disclosure is used.

### BEST MODE FOR CARRYING OUT THE INVENTION

The attached drawings for illustrating exemplary embodiments are referred to in order to gain a sufficient understanding of the merits and features thereof, and methods for accomplishing the merits and features thereof.

The present disclosure may, however, be embodied in many different forms and should not be construed as being limited to the embodiments set forth herein; rather, these embodiments are provided so that this disclosure will be thorough and complete, and will fully convey the concept of the disclosure to those skilled in the art. The scope of the present disclosure is only defined by the appended claims. Throughout the specification, like reference numerals or characters refer to like elements.

Embodiments of the present disclosure will now be described more fully with reference to the accompanying drawings such that one of ordinary skill in the art to which the present disclosure pertains may easily execute the present disclosure. The present disclosure may, however, be embodied in many different forms and should not be construed as being limited to the embodiments set forth herein. The same reference numerals are assigned to similar parts throughout the specification.

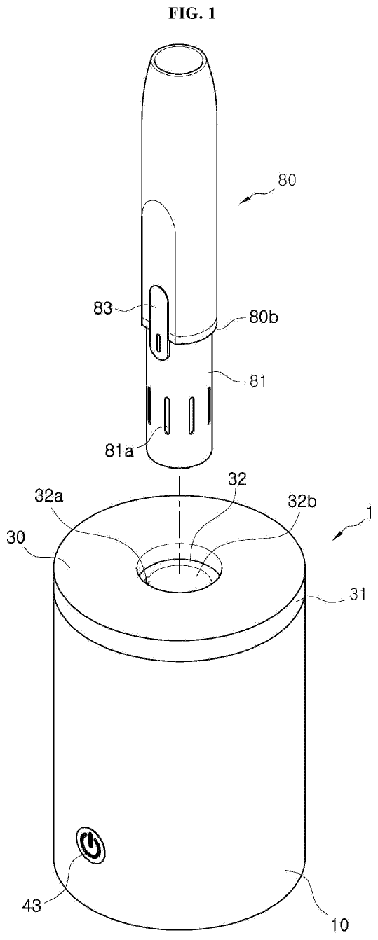

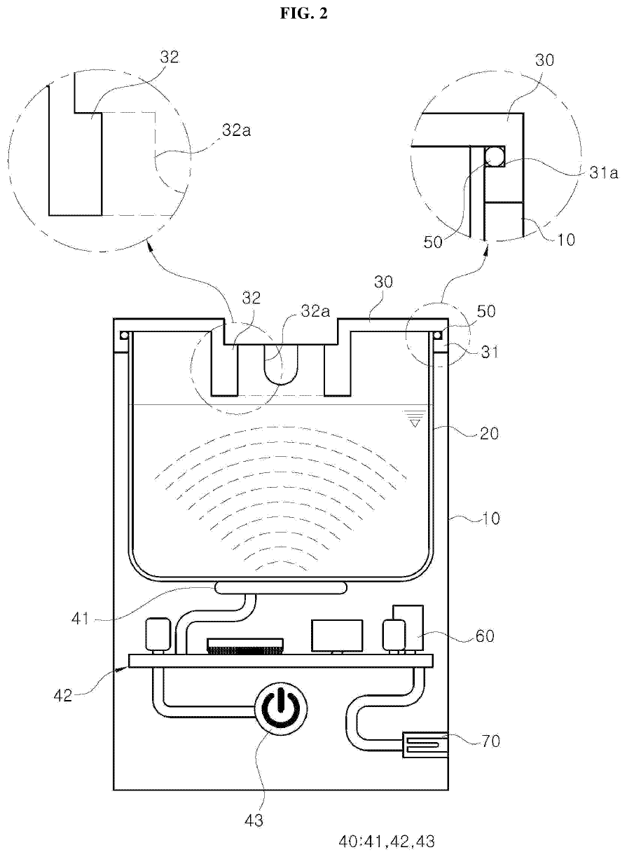

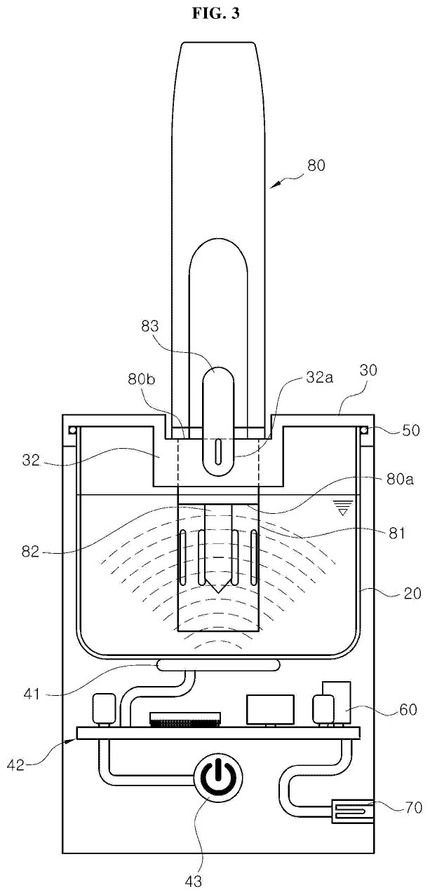

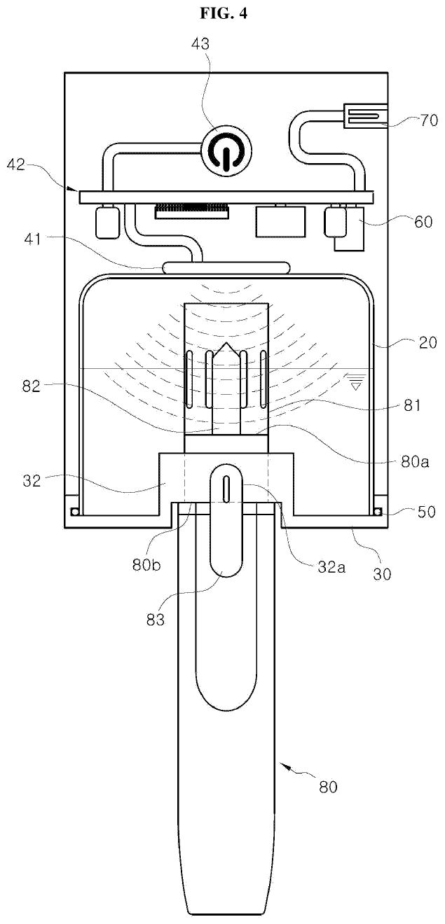

FIG. 1 is a perspective view of an electronic cigarette automatic cleaning apparatus according to the present disclosure, FIG. 2 is a view illustrating the inside of the electronic cigarette automatic cleaning apparatus according to the present disclosure, and FIGS. 3 and 4 are views illustrating states in which the electronic cigarette automatic cleaning apparatus according to the present disclosure is used.

An electronic cigarette automatic cleaning apparatus 1 according to the present disclosure is a product capable of automatically, rapidly, and thoroughly cleaning a holder 80 of an electronic cigarette device without using cleaning tools such as a foreign matter removing brush or cotton swab.

To this end, the electronic cigarette automatic cleaning apparatus 1 according to the present disclosure may include a main body 10, a cleaning tub 20, a cover 30, and an ultrasound generation portion 40.

An electronic cigarette device to be cleaned by the present disclosure may be, for example, an IQOS or a WHYQOS or CHIQOS compatible with an IQOS cigarette. An IQOS will now be illustrated and described as an example.

In addition, the present disclosure involves an apparatus that cleans a holder 80 for holding a cigarette, from among the components of a IQOS cigarette, as well as a cleaning method of the apparatus.

The main body 10 has an upper surface that is open. An accommodating space in which the cleaning tub 20 is accommodated, and an installation space in which the ultrasound generation portion 40 is installed are defined in a vertical direction within the main body 10.

The main body 10 may be manufactured to have a size that is easy for a user to grasp. The outer surface of the main body 10 may be finished with rubber or silicon to prevent slipping during grasping, or an anti-slip pad made of rubber or silicone may be attached to the outer surface of the main body 10.

The cleaning tub 20 is configured to fit within the main body 10 and contain a cleaning material capable of cleaning a cleanable portion of the holder 80.

The cleanable portion of the holder 80 may comprise a heating portion 82, an insertion tube 81 (which surrounds heating portion 82), and a bottom surface 80a of the holder 80 from which the heating portion 82 protrudes.

The cleaning tub 20 has an upper surface that is open, and a storage space in which the cleaning material is stored.

The cleaning tub 20 is held within the accommodating space of the main body 10. A portion of the upper side of the cleaning tub 20 protrudes past the upper surface of main body 10 and is accommodated within the cover 30. (See FIG. 2)

The cleaning material stored in the cleaning tub 20 may be water, cleaning alcohol, or any other available cleaning fluid.

The cleaning tub 20 may be formed to have a size and a shape that conforms to the accommodating space of the main body 10, and may be removable so that when the cleaning material is contaminated, a user may easily replace the contaminated cleaning material with a new cleaning material.

Although not shown in the drawings, the interfacing surfaces of the cleaning tub and main body may be textured to aid in holding the cleaning tub in the main body. For example, the exterior of the sidewall of the cleaning tub and the inside surface of the accommodating space can have a roughened surface to aid in a friction fit. In one embodiment, the textured surface comprises a set of spirals meshing with each other. The spirals may have a semicircular or semielliptical cross-section and may be a set of parallel rings, or the spirals may be helical.

The cover 30 covers the top of the cleaning tub 20 such that the cleaning tub 20 has a minimum open portion. Cover 30 is designed to reduce spillage of the cleaning material and also support the holder 80 when the cleanable portion of the holder 80 is cleaned.

Cover 30 comprises a through hole 32b enabling the cleanable portion of the holder 80 to pass therethrough to be immersed in the cleaning material. To support the holder 80 during use, a support step 32 is formed in cover 30 around through hole 32b. Through hole 23b is sized and otherwise configured such that only the desired portion of holder 80 to be cleaned can fit therethrough. A support groove 32a defines a cavity in cover 30, such as within through hole 32b, configured to accommodate at least a portion of the power button 83 of holder 80.

Accordingly, as shown in FIG. 3, when the holder 80 is placed in the apparatus, the insertion tube 81 is passed through the through hole 32b, the end jaw 80b is stopped on the support step 32, and thus only the insertion tube 81, the heating portion 82, and the bottom surface 80a of the holder 80 are immersed in the cleaning material.

During cleaning, due to a plurality of long holes 81a formed in the insertion tube 81, the cleaning material may flow into the insertion tube 81 through the long holes 81a and thus may evenly contact the heating portion 82 and the bottom surface 80a.

In order to reduce spillage, cover 30 is configured to form a seal along with cleaning tub 20. As shown in FIG. 2, the outer edge of cover 30 comprises a flange 31 extending at a right angle from the outer circumference of the cover 30. Flange 31 is configured to extend and surround the portion of the cleaning tube 20 which extends beyond main body 10. A fixing groove 31a is formed in the inner circumference of flange 31 and is configured to hold a seal 50. Seal 50 may be an O-ring formed of rubber, silicon, or any other material suitable for contact with the cleaning material. When cover 30 is installed, flange 31 surrounds a portion of the cleaning tub 20 and seal 50 is compressed against cleaning tub 20 and fixing groove 31a, which prevents spillage of the cleaning material. Spillage of the cleaning material may be prevented even when the electronic cigarette automatic cleaning apparatus 1 is shaken from side to side or is flipped 180 degrees.

Accordingly, when the electronic cigarette automatic cleaning apparatus 1 and the holder 80 erected upright as shown in FIG. 3 is flipped 180 degrees as shown in FIG. 4, the cleaning material may completely adhere to the entire cleanable portion of the holder 80.

The ultrasound generation portion 40 is configured to aid in removing foreign materials, such as cigarette ash and dust, attached to the insertion tube 81, the heating portion 82, and the bottom surface 80a.

To this end, various ultrasound generators that generates ultrasonic waves on the cleaning material may be used as the ultrasound generation portion 40, and the type and characteristics of the ultrasound generation portion 40 do not restrict the present disclosure.

For example, the ultrasound generation portion 40 may include one or more ultrasonic vibrators 41, and the ultrasonic vibrators 41 are provided in the installation space of the main body 10 and contact the lower surface of the cleaning tub 20.

The ultrasonic vibrators 41 are electrically connected to a driving circuit 42 provided in the installation space, and the driving circuit 42 is electrically connected to a power button 43.

The power button 43 turns on or off an operation of the ultrasound generation portion 40 and is exposed to the outside of the main body 10 so as to be easily manipulated by a user.

A charging portion 60 for charging the ultrasonic vibrators 41 is further provided on the driving circuit 42 and includes a charging terminal 70 to which a typical connection cable is connectable.

The charging terminal 70 is exposed to the outside of the main body 10 so that the connection cable may be easily connected to the charging terminal 70. The charging terminal 70 supplies electric energy from a connected power source to the battery of the electronic cigarette automatic cleaning apparatus 1, so that the battery of the electronic cigarette automatic cleaning apparatus 1 may be charged. The present disclosure is not particularly limited thereto, and includes everything well known in the art.

When the ultrasound generation portion 40 is operated by pressing the power button 43, the ultrasonic vibrators 41 generate ultrasonic waves to make the cleaning material within the cleaning tub 20 vibrate at a high speed, and this vibration detaches foreign materials, such as ash, powder, and dust of a cigarette, from the inner surface and outer surface of the insertion tube 81, the surface of the heating portion 82, and the bottom surface 80a of the holder 80.

In some cases, the ultrasonic vibrators 41 may be provided within the cleaning tub 20.

The locations of the ultrasonic vibrators 41 and the number of ultrasonic vibrators 41 may vary according to required conditions and design specifications. For example, when a plurality of ultrasonic vibrators 41 are applied, the plurality of ultrasonic vibrators 41 may be provided apart from each other on the outer surface of the cleaning tub 20, the inner surface of the cleaning tub 20, or both the outer surface and the inner surface of the cleaning tub 20.

In this case, ultrasonic vibration occurs evenly in the entire cleaning material, and thus efficiency of cleaning the insertion tube 81, the heating portion 82, and the bottom surface 80a may be increased.

As described above, the electronic cigarette automatic cleaning apparatus 1 according to the present disclosure may automatically, quickly, and thoroughly clean the insertion tube 81, the heating portion 82, and the bottom surface 80a on which foreign materials such as cigarette powder, cigarette ash, and dust are intensively accumulated, thereby improving the convenience of a smoker who uses an electronic cigarette device. In addition, the electronic cigarette automatic cleaning apparatus 1 according to the present disclosure may completely remove the foreign materials through a cleaning material instead of detaching foreign materials through a conventional foreign material removal brush, thereby enabling clean usage. Moreover, the electronic cigarette automatic cleaning apparatus 1 according to the present disclosure may prevent a smoker from inhaling harmful ingredients generated from cigarette powder, cigarette ash, or the like, thereby preventing an adverse influence upon the smoker's health.

One of ordinary skill in the art to which the present disclosure pertains will understand that various changes in form and details may be made therein without departing from the spirit and scope of the present disclosure. Thus, the above-described embodiments should be considered in descriptive sense only and not for purposes of limitation. The scope of the present disclosure is indicated by the scope of the claims to be described later rather than the above detailed description, and all changes or modified forms derived from the meaning and scope of the claims and the concept of equivalents thereof should be interpreted as being included in the scope of the present disclosure.

1. An electronic cigarette automatic cleaning apparatus comprising:
a main body having an accommodating space therein;
a cleaning tub provided in the accommodating space of the main body, the cleaning tub storing a cleaning material for cleaning an electronic cigarette device;
a cover coupled to the cleaning tub to cover a top of the cleaning tub;
an ultrasound generation portion provided in the main body and configured to emit ultrasonic waves to the cleaning material to separate a foreign material from the electronic cigarette device,
a support step supporting the electronic cigarette device is recessed in a center portion of the cover, and
a through hole in an inner portion of the support step through which a cleanable portion of the electronic cigarette device is inserted to be immersed in the cleaning material.

2. The electronic cigarette automatic cleaning apparatus of claim 1, wherein a packing is provided on an outer surface of the cleaning tub, and a fixing groove into which the packing is inserted and fixed is formed in an inner surface of the cover.

3. The electronic cigarette automatic cleaning apparatus of claim 1, wherein a support groove for supporting a power button included in the electronic cigarette device is formed in an inner surface of the support step.

4. The electronic cigarette automatic cleaning apparatus of claim 1, further comprising a charging portion provided in the main body and having a charging terminal to charge the ultrasound generation portion.

5. The electronic cigarette automatic cleaning apparatus of claim 1, wherein the cleaning tub is removable from the accommodating space of the main body.

6. The electronic cigarette automatic cleaning apparatus of claim 5, wherein the at least one of the cleaning tub and the accommodating space comprise a textured surface to aid in retaining the cleaning tub in the accommodating space.

7. The electronic cigarette automatic cleaning apparatus of claim 6, wherein the textured surface comprises a set of spirals.

8. The electronic cigarette automatic cleaning apparatus of claim 1, wherein the cleaning material is water.

9. The electronic cigarette automatic cleaning apparatus of claim 1, wherein the cleaning material is a cleaning alcohol.

10. An electronic cigarette automatic cleaning apparatus comprising:
a main body having an accommodating space therein;
a cleaning tub provided in the accommodating space of the main body, the cleaning tub storing a cleaning material for cleaning an electronic cigarette device;
an ultrasound generation portion provided in the main body and configured to emit ultrasonic waves to the cleaning material to separate a foreign material from the electronic cigarette device, and
a cover coupled to the cleaning tub to cover a top of the cleaning tub, the cover comprising:
a through hole through which a cleanable portion of the electronic cigarette device is inserted to be immersed in the cleaning material,
a flange extending from the outer edge of the cover for accommodating a portion of the cleaning tub,
a fixing groove in the flange, and
a seal in the fixing groove.

11. The electronic cigarette automatic cleaning apparatus of claim 10, wherein the cleaning tub is removable from the accommodating space of the main body.

12. The electronic cigarette automatic cleaning apparatus of claim 11, wherein the at least one of the cleaning tub and the accommodating space comprise a textured surface to aid in retaining the cleaning tub in the accommodating space.

13. The electronic cigarette automatic cleaning apparatus of claim 12, wherein the textured surface comprises a set of spirals.

14. The electronic cigarette automatic cleaning apparatus of claim 10, further comprising:
a battery, and
a charging portion provided in the main body for facilitating charging to the battery.

15. The electronic cigarette automatic cleaning apparatus of claim 10, wherein the cleaning material is water.

16. The electronic cigarette automatic cleaning apparatus of claim 10, wherein the cleaning material is a cleaning alcohol.

17. An electronic cigarette automatic cleaning apparatus comprising:
a main body having an accommodating space therein;
a cleaning tub provided in the accommodating space of the main body, the cleaning tub storing a cleaning material for cleaning an electronic cigarette device;
an ultrasound generation portion provided in the main body and configured to emit ultrasonic waves to the cleaning material to separate a foreign material from the electronic cigarette device, and
a cover coupled to the cleaning tub to cover a top of the cleaning tub, the cover comprising:
a support step for supporting the electronic cigarette device is recessed in a center portion of the cover,
a support groove for accommodating a power button of the electronic cigarette device,
a through hole in an inner portion of the support step through which a cleanable portion of the electronic cigarette device is inserted to be immersed in the cleaning material,
a flange extending from the outer edge of the cover for accommodating a portion of the cleaning tub,
a fixing groove in the flange, and
a seal in the fixing groove.

18. The electronic cigarette automatic cleaning apparatus of claim 17, further comprising:
a battery, and
a charging portion provided in the main body for facilitating charging to the battery.

19. The electronic cigarette automatic cleaning apparatus of claim 17, wherein the cleaning material is water.

20. The electronic cigarette automatic cleaning apparatus of claim 17, wherein the cleaning material is a cleaning alcohol.
